(19) United States
(12) Patent Application Publication
(10) Pub. No.: US 2026/0315145 A1
(43) Pub. Date: Oct. 15, 2026

(54) LASER TIP WITH CAPILLARY EFFECT AND PASSIVE SAFETY MECHANISM

(71) Applicants: Derek Djeu, Tampa, FL (US); Garret Djeu, Annandale, VA (US)

(72) Inventors: Derek Djeu, Tampa, FL (US); Garret Djeu, Annandale, VA (US)

(21) Appl. No.: 19/431,192

(22) Filed: Dec. 23, 2025

Related U.S. Application Data
(60) Provisional application No. 63/787,769, filed on Apr. 12, 2025.

(51) Int. Cl. A61B 18/26; A61B 18/20; A61B 18/22; A61C 1/00

(52) U.S. Cl. CPC A61B 18/26; A61C 1/0046; A61B 2018/206; A61B 2018/2222; A61B 2018/2261; A61B 2018/263

(57) ABSTRACT

A laser tip assembly includes a capillary tube having an open distal end and a single crystal optical fiber positioned within the capillary tube. The single crystal optical fiber causes guided light to spread with angular divergence and is recessed from the open distal end. A gap between the single crystal optical fiber and capillary tube draws liquid toward the fiber via capillary effect. This directs laser energy into liquid rather than surrounding tissue, creating cavitation bubbles while preventing thermal damage. The angular divergence provides additional safety whereby if no liquid is present, spreading light contacts capillary tube walls rather than tissue. A method includes positioning the assembly adjacent to a treatment area, allowing capillary effect to draw liquid into the tube, and delivering laser energy through the fiber to create cavitation bubbles for medical treatment.

### CROSS REFERENCE TO RELATED APPLICATIONS

This application claims the benefit of U.S. Provisional Patent Application 63/787,769, filed on Apr. 12, 2025, and incorporated herein by reference.

### STATEMENT REGARDING FEDERALLY SPONSORED RESEARCH AND DEVELOPMENT

Not Applicable.

### FIELD OF THE DISCLOSURE

The present disclosure generally relates to laser medical instruments, and more particularly to a laser tip assembly used in medical and dental procedures for delivering laser energy through a liquid medium to a treatment site.

### BACKGROUND

Laser systems are increasingly employed in medical and dental procedures for cutting, ablation, and cleaning of biological tissue. In endodontic and other minimally invasive treatments, optical fibers are used to deliver laser energy to confined regions such as root canals, periodontal pockets, or other cavities containing fluid. However, precise control of laser-tissue interaction remains a significant challenge. When the fiber tip directly contacts or is positioned too close to the tissue, localized overheating and carbonization can occur, leading to unintended thermal damage to surrounding soft or hard tissue.

Various approaches have been developed to reduce thermal injury, including modulation of pulse duration, adjustment of laser power, and the use of fluid irrigation around the treatment site. Nonetheless, these methods often rely on active control or operator skill and may not reliably ensure that liquid remains in the optical path of the laser. When liquid is displaced or insufficiently present, the laser energy can directly strike the tissue, increasing the risk of injury.

Additionally, many existing fiber-based laser delivery devices utilize flat or flush fiber tips that emit laser radiation directly into air or tissue. Such designs provide limited passive safety mechanisms and depend on precise manual positioning by the clinician. In high-power laser applications, even minor misalignment or absence of liquid can cause excessive heat accumulation at the distal tip.

Accordingly, there remains a need for a laser delivery assembly that addresses the fundamental limitations of existing systems. The prior art fails to provide reliable passive mechanisms for maintaining liquid in the laser path, lacks inherent safety features when liquid is absent, and requires constant operator vigilance to prevent thermal damage. An ideal laser delivery system would passively maintain a liquid interface at the laser emission point without relying on active control, would provide multiple independent safety mechanisms to prevent direct laser-tissue contact, and would automatically ensure that laser energy interacts primarily with liquid to generate safe cavitation effects rather than causing thermal damage to surrounding tissue. Such a system would eliminate the dependence on operator skill and positioning accuracy while providing consistent protection even in challenging anatomical environments where liquid levels may vary.

### SUMMARY OF THE DISCLOSURE

The present invention is a laser tip assembly that includes a capillary tube having an open distal end and an optical waveguide positioned within the capillary tube. The optical waveguide causes guided light from the proximal end to spread and exit the distal end with an increased angular divergence, providing a unique safety mechanism. In some cases, for example the optical waveguide is a single crystal fiber, the angular divergence of the light will increase with the distance of propagation. The optical waveguide is recessed from the open distal end of the capillary tube. A gap is formed between the optical waveguide and the capillary tube, where the gap is configured to draw liquid into the capillary tube toward the single crystal optical fiber via a capillary effect. This configuration ensures that laser energy is directed into the liquid rather than surrounding tissue, creating cavitation bubbles for medical procedures while preventing thermal damage to soft or hard tissue.

When the optical waveguide is a single crystal fiber, the single crystal optical fiber may have various cross-sectional shapes such as hexagonal or oblong and may be tapered. The capillary tube may be constructed of metal and may be closed at one end to limit the capillary effect. When the optical waveguide is a single crystal fiber, the capillary tube can be thin enough to bend with the single crystal optical fiber and may contact vertices of the single crystal optical fiber. The gap may extend to both ends of the capillary tube to allow maximum capillary action. The angular divergence property of the optical waveguide provides an additional safety feature whereby if no liquid is present in the capillary tube, a material amount of the spreading light contacts the inner walls of the capillary tube rather than surrounding tissue, and the remaining light that still does pass through the distal end is not focused and thus less likely to damage tissue.

The invention also includes a method of delivering laser energy by positioning the laser tip assembly adjacent to a treatment area containing liquid, allowing the capillary effect to draw the liquid into the capillary tube toward the recessed optical waveguide, and delivering laser energy through the optical waveguide into the liquid within the capillary tube. The laser energy creates cavitation bubbles in the liquid that can break up soft tissue or hard tissue and provide disinfection in medical procedures such as endodontics.

The present invention addresses the drawbacks of the prior art by providing a laser delivery assembly that passively maintains a liquid interface at the laser emission point through a capillary effect and provides dual safety mechanisms through the unique optical properties of the optical waveguide, such as single crystal fibers, tapered waveguides and waveguides with rounded ends. The invention ensures that laser energy interacts primarily with the liquid to generate cavitation effects while minimizing thermal exposure of surrounding tissue, eliminating the need for active control or precise manual positioning by the clinician. The capillary action created by the gap between the recessed optical waveguide and the capillary tube automatically draws liquid into the laser path, while the angular divergence property of the optical waveguide ensures that any light not absorbed by liquid will spread and materially contact the capillary tube walls rather than tissue, while the remaining light that passes through the distal end will not be focused and less likely to damage tissue. This provides a reliable dual passive safety mechanism that prevents direct laser-tissue contact even in high-power applications. Other features and advantages of the present invention will become apparent from the following more detailed description, taken in conjunction with the accompanying drawings, which illustrate, by way of example, the principles of the invention.

### DESCRIPTION OF THE DRAWINGS

FIG. 1 illustrates a perspective view of a laser tip assembly, consistent with an illustrative embodiment;

FIG. 2 is a cross-sectional view of the laser tip assembly, consistent with an illustrative embodiment;

FIG. 3 illustrates a cross-sectional view of the laser tip assembly showing an oblong-cross-section of a single crystal optical fiber, metal capillary tube, and a resulting gap that allows for capillary action;

FIG. 4 is a side view of an example laser tip assembly with a tapered optical waveguide;

FIG. 5 shows a side view of an example laser tip assembly where the optical fiber is tapered optical waveguide that is closed at the proximal end; and

FIG. 6 shows a side view of an example laser tip assembly where the optical fiber is rounded at a distal end; and

FIG. 7 presents an illustrative process of providing laser energy via a laser tip assembly;

### DETAILED DESCRIPTION OF THE PREFERRED EMBODIMENT

Illustrative embodiments of the disclosure are described below. The following explanation provides specific details for a thorough understanding of and enabling description for these embodiments. One skilled in the art will understand that the disclosure may be practiced without such details. In other instances, well-known structures and functions have not been shown or described in detail to avoid unnecessarily obscuring the description of the embodiments.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list. When the word “each” is used to refer to an element that was previously introduced as being at least one in number, the word “each” does not necessarily imply a plurality of the elements but can also mean a singular element. When the words “substantially” or “about” are used, if a quantitative measurement is necessary, within 95% of “complete” or “exact” should be considered the meaning. When the word “materially” is used, if a quantitative measurement is necessary, within 5% of “complete” or “exact” should be considered the meaning. The term “the disclosure” or “the present disclosure” should always be construed as “an embodiment of the disclosure.”

This present disclosure relates to a laser tip assembly featuring a single crystal optical fiber that is deliberately recessed within a metal capillary tube, where the gap between the fiber and tube creates a capillary effect to draw liquid into the capillary tube toward the recessed fiber tip. The single crystal optical fiber causes guided light to spread with angular divergence that increases with distance of propagation, providing a dual safety mechanism. The capillary action ensures that laser energy is directed into the liquid rather than surrounding tissue, creating cavitation bubbles for medical procedures while preventing thermal damage to soft or hard tissue. As used herein the capillary effect, sometimes referred to as capillarity or capillary action, relates to a fluid-dynamic phenomenon where a liquid spontaneously rises (or falls) in a narrow space—like a thin tube, a small pore, or between closely spaced fibers—without the assistance of, and sometimes against, external forces like gravity. This action occurs mainly because of the interplay of two molecular forces: (i) cohesion—related to the attraction between molecules of the liquid (e.g., water-water), and (ii) adhesion—the attraction between molecules of the liquid and the solid surface (e.g., water-container). This safety mechanism provides enhanced protection during laser medical procedures such as endodontics by passively positioning liquid in the laser beam path. In certain laser medical procedures, such as in endodontics, a laser pulse can be used to create a cavitation bubble in water or liquid. The cavitation bubble can break up soft tissue and also disinfect. The laser pulse can be delivered to the water or liquid by an optical fiber tip or optical waveguide, made from metal, silica, quartz, or sapphire.

Single crystal optical fibers, particularly sapphire fibers, exhibit unique optical properties that distinguish them from traditional silica-based optical fibers. The Numerical Aperture of a sapphire fiber is not well defined due to its typical hexagonal or oblong cross-sectional shape, surface characteristics, and uniaxial crystal structure. Unlike traditional fibers that maintain collimated light output, single crystal fibers cause the angular spread of guided light to increase with distance of propagation. This means that even if collimated laser light enters the single crystal fiber, the light exiting the fiber spreads out more like a flashlight rather than maintaining a focused laser beam. This spreading occurs because large angle rays make more bounces at the fiber walls per unit length of propagation, resulting in increased scattering. This optical property provides an important safety feature: if no liquid is present in the capillary tube to create cavitation bubbles, a material amount of the spreading light will contact the inner walls of the capillary tube rather than directly striking surrounding tissue, thereby preventing thermal damage to the patient.

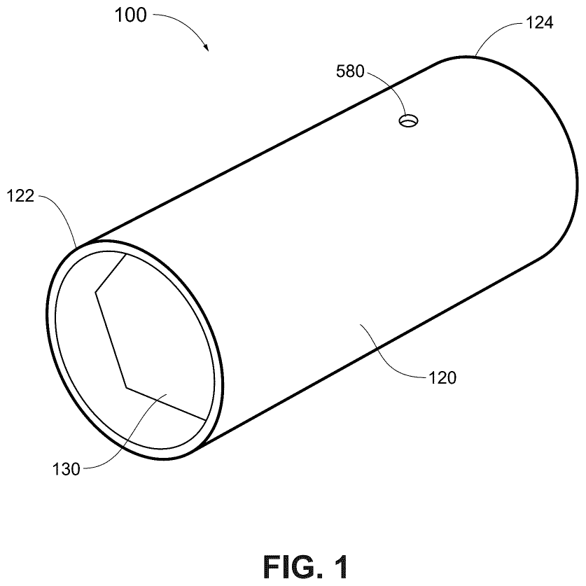

Reference now is made to FIG. 1, which illustrates a perspective view of a laser tip assembly 100, consistent with an illustrative embodiment. The laser tip assembly 100 includes a capillary tube 120 having an open distal end 122 and a proximal end 124. In one embodiment, the capillary tube 120 is constructed of a metal. For example, a metal is used instead of other materials (e.g., plastics) because it is a good thermal conductor. The metallic composition of the capillary tube functions as a heat sink to dissipate localized heat generated near the distal tip during laser emission. In one embodiment, the capillary tube 120 comprises a metal that is thin enough to bend with the single crystal optical fiber 130, also referred to herein as a waveguide 130. Stated differently, the metal capillary tube has a wall thickness selected such that its flexibility substantially matches that of the single crystal optical fiber, thereby enabling the assembly to bend as a unit when navigating curved anatomical structures. Such thin-walled metal capillary allows the entire laser tip assembly 100 to bend together during navigation through curved anatomical pathways, such as root canals, blood vessels, or other narrow, tortuous cavities. If the capillary tube were rigid or significantly thicker than the fiber, it would restrict movement and risk fiber breakage and/or misalignment. By matching the flexibility, the metal capillary tube can protect the single crystal optical fiber without compromising steerability. Such flexibility also facilitates uniform energy delivery even when the assembly is bent, ensuring the distal end 122 remains coaxial.

There is a single crystal optical fiber 130 positioned within the capillary tube 120. The single crystal optical fiber 130 is recessed from the open distal end 122 of the capillary tube 120. In various embodiments the single crystal optical fiber 130 can include materials such as sapphire (Al2O3) or yttrium aluminum garnet (YAG) that can be pulled as single-crystal fibers. Such single-crystal fibers can be used together with high-power laser delivery, since they are stronger and have higher damage thresholds than glass fibers. In one embodiment, the single crystal optical fiber 130 comprises silica.

The distance of recession into the capillary tube is optimized to allow the liquid to be drawn up in the capillary tube and contact the end of the single crystal fiber or come close to contacting the end of the single crystal fiber. The distance of the recession is also selected to allow the laser beam to spread out due to the angular divergence property of the single crystal fiber, such that if no liquid is present, the spreading light contacts the inner walls of the capillary tube rather than exiting toward surrounding tissue.

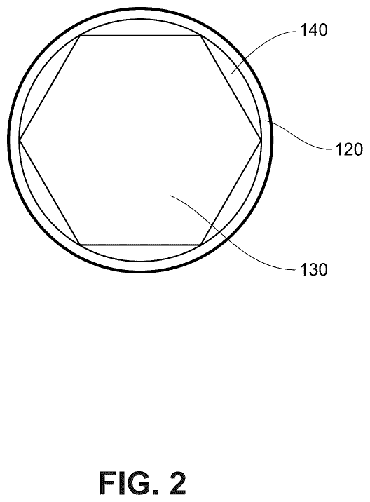

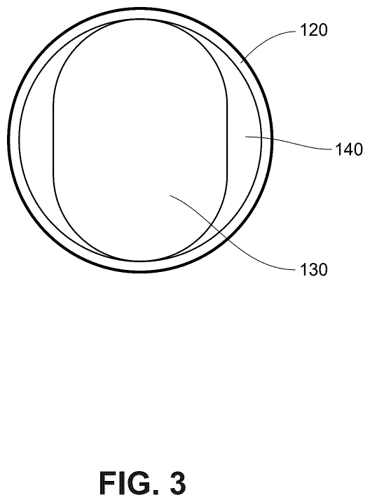

FIG. 2 is a cross-sectional view of the laser tip assembly, consistent with an illustrative embodiment. There is a gap 140, formed between the single crystal optical fiber 130 and the capillary tube 120, wherein the gap 140 is configured to draw a liquid into the capillary tube 120 toward the single crystal optical fiber 130 via a capillary effect. As illustrated in FIG. 2, in one embodiment, the single crystal optical fiber 130 has a hexagonal cross-section, while it will be understood that other polygonal cross-section as well as oblong profiles are supported by the teachings herein as well. In this regard, FIG. 3 illustrates a cross-sectional view of the laser tip assembly showing an oblong-cross-section of a single crystal optical fiber, metal capillary tube 120, and a resulting gap 140 that allows for capillary action. In one embodiment, the inner surface of the capillary tube 120 contacts one or more vertices of the single crystal optical fiber 130. The gap 140 can extend to both ends of the capillary tube 120.

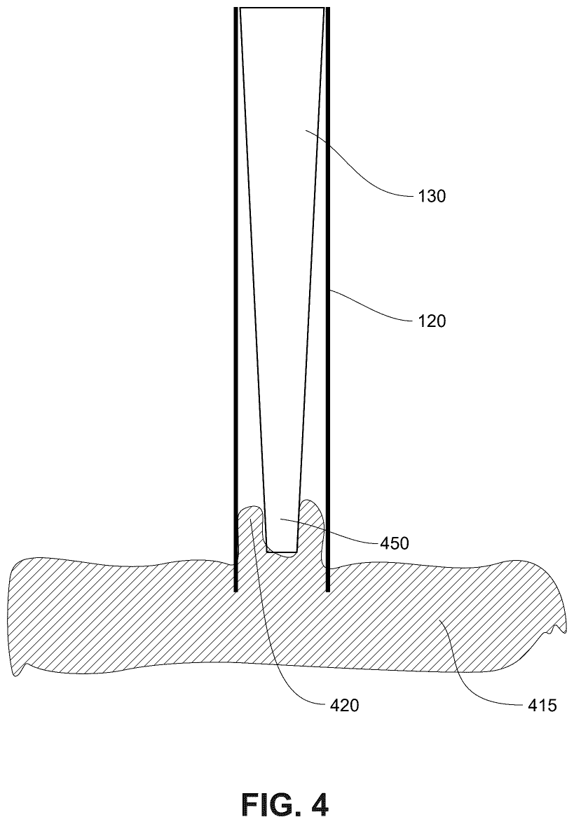

FIG. 4 is a side view of an example laser tip assembly with a tapered optical waveguide130. For example, the tapered end 450 allows more liquid 415 to surround the tip of the single crystal waveguide, as illustrated by the elevated liquid 420. The air gap 140 extends to both ends of the capillary tube to allow for improved capillary action. The surrounding liquid level 415 is lower than the capillary tube liquid level 420, since the capillary action draws the liquid up into the capillary tube 120. Accordingly, the tapered waveguide 130 increases the energy density at the output tip while also enhancing the light scattering effect. The result is more efficient cavitation bubble production in the fluid while maintaining the safety benefit of light spreading when no liquid is present. For example, in medical applications, this allows more precise energy delivery while maintaining lower total power, reducing the risk of excessive heating. Thus, the tapered geometry of the waveguide 130 can increase the intensity of emitted laser radiation at the distal end, thereby improving cavitation efficiency while potentially enabling operation at lower power levels. In one embodiment, the tapering of the waveguide 130 provides a variable-width annular gap that promotes continuous capillary flow of liquid toward the recessed optical fiber tip. The optical waveguide could be made of a material such as sapphire, YAG or silica-based materials.

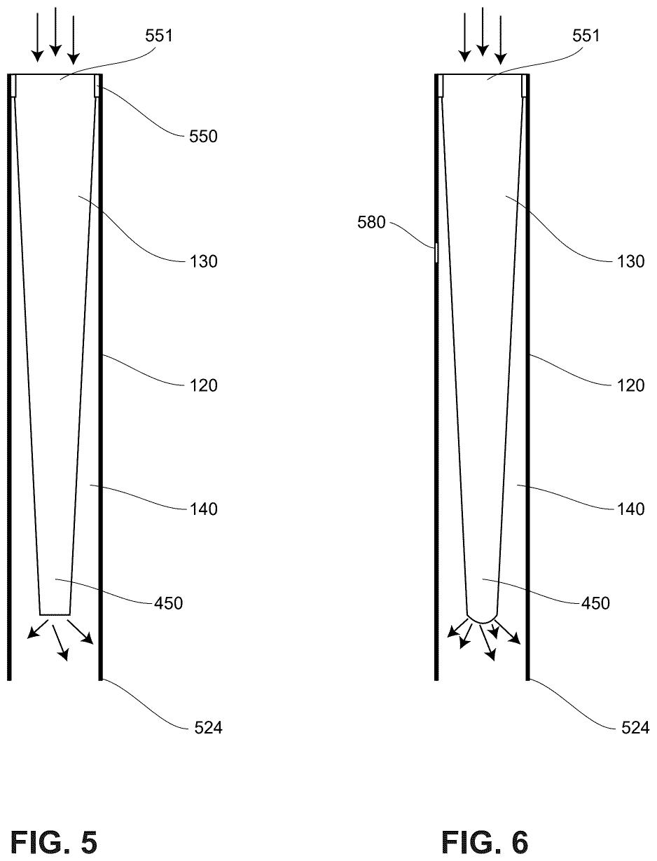

FIG. 5 shows a side view of an example laser tip assembly where the optical waveguide is tapered, and the capillary tube is closed at a proximal end 551 and open at a distal end 524. The figure illustrates coherent light entering at a top of the optical waveguide and scattered light exiting at a bottom end, demonstrating the angular divergence property that provides the safety mechanism. For example, the tapered end 450 can allow more liquid to surround the tip of the optical waveguide 130. In one embodiment, the air gap 140 is closed a proximal end 551 of the capillary tube 120 by an adhesive 550 to limit the capillary action for the purpose of controlling the liquid level 420. Stated differently, the capillary tube 120 is closed at a proximal end 551 to limit the capillary effect. FIG. 6 illustrates a similar embodiment but wherein the tapered end 450 is rounded, providing even more scattering of light. In another embodiment, the capillary tube 120 may include an aperture 580 (FIGS. 1 and 6) to expose the gap 140 to ambient pressure, thereby controlling the capillary action. The aperture 580 may be positioned along the length of the capillary tube 120 to allow air to enter or exit the gap 140, which can regulate the extent of liquid drawn into the capillary tube via the capillary effect.

Accordingly, in embodiments, the teachings herein provide a new laser tip assembly structure, using a metal capillary tube around an optical waveguide that is recessed in the metal capillary tube. The optical waveguide provides a dual safety mechanism: the capillary tube draws liquid into the capillary tube so that the end of the single crystal fiber is immersed in the liquid, and the angular divergence property of this particular optical waveguide causes light to spread and contact the capillary tube walls rather than tissue when no liquid is present. This helps to ensure that the laser energy is directed into the liquid to create cavitation bubbles, as opposed to being directed into nearby soft or hard tissue which may create thermal damage. The capillary tube can be thin enough to bend with the single crystal fiber and positioned so that it can contact vertices of the single crystal fiber. The capillary tube can be open at both ends to allow for maximum capillary effect or can be closed at one end to limit the capillary effect.

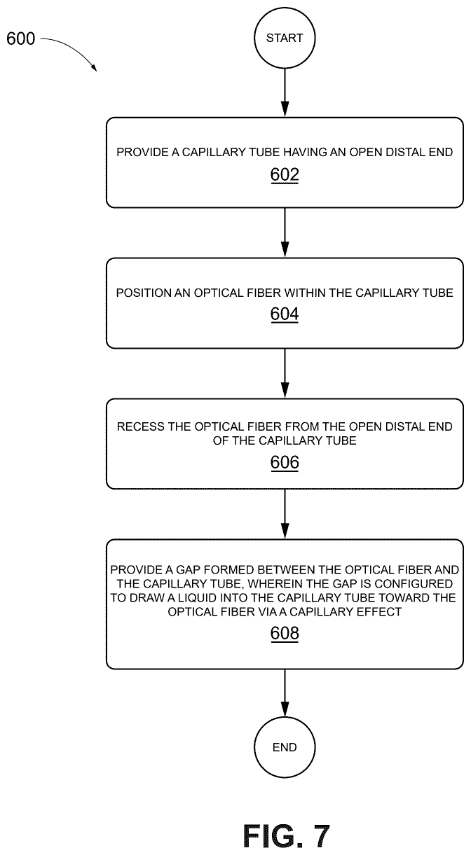

With the foregoing overview of the example architectures of a laser tip assembly, it may be helpful now to consider a high-level discussion of an example process. To that end, FIG. 7 presents an illustrative process 600 of providing laser energy via a laser tip assembly 100. The process will be discussed with reference to FIGS. 1 and 2. At block 602, a capillary tube 120 is provided having an open distal end 122. At block 604, an optical waveguide 130 is positioned within the capillary tube 120, wherein the optical waveguide causes guided light to spread with angular divergence that increases through the optical waveguide. At block 606, the optical waveguide 130 is recessed from the open distal end 122 of the capillary tube 120. At block 608 a gap 140 is formed between the optical waveguide 130 and the capillary tube 120. The gap 140 is configured to draw a liquid 415 into the capillary tube 120 toward the optical waveguide 130 via a capillary effect. The capillary tube can be constructed of a metal.

In one example, the laser tip assembly 100 can be positioned adjacent a treatment area including a liquid 415. Positioning the laser tip assembly 100 can include bending the capillary tube 120 with the optical waveguide 130. Liquid 415 can be drawn into the capillary tube 120 toward the optical waveguide. Laser energy can be delivered through the optical waveguide 130 into the liquid 415 within the capillary tube 120. As explained above, a cavitation bubble can be created in the liquid 415 with the laser energy. Creating the cavitation bubble is operative to break up a soft tissue and/or provide disinfection.

While a particular form of the disclosure has been illustrated and described, it will be apparent that various modifications can be made without departing from the spirit and scope of the disclosure. For example, while certain shapes of cross-sections of the optical waveguide 130 are illustrated, it will be understood that other shapes are supported as well. Similarly, different materials for the capillary tube 120 can be used, as appropriate for different environments, based on the heat dissipation and other relevant factors. Accordingly, it is not intended that the disclosure be limited, except as by the appended claims.

Particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure.

The above detailed description of the embodiments of the disclosure is not intended to be exhaustive or to limit the disclosure to the precise form disclosed above or to the particular field of usage mentioned in this disclosure. While specific embodiments of, and examples for, the disclosure are described above for illustrative purposes, various equivalent modifications are possible within the scope of the disclosure, as those skilled in the relevant art will recognize. Also, the teachings of the disclosure provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the disclosure can be modified, if necessary, to employ the systems, functions, and concepts of the various references described above to provide yet further embodiments of the disclosure.

Changes can be made to the disclosure in light of the above “Detailed Description.” While the above description details certain embodiments of the disclosure and describes the best mode contemplated, no matter how detailed the above appears in text, the disclosure can be practiced in many ways. Therefore, implementation details may vary considerably while still being encompassed by the disclosure disclosed herein. As noted above, particular terminology used when describing certain features or aspects of the disclosure should not be taken to imply that the terminology is being redefined herein to be restricted to any specific characteristics, features, or aspects of the disclosure with which that terminology is associated.

While certain aspects of the disclosure are presented below in certain claim forms, the inventor contemplates the various aspects of the disclosure in any number of claim forms. Accordingly, the inventor reserves the right to add additional claims after filing the application to pursue such additional claim forms for other aspects of the disclosure.

What is claimed is:

1. A laser tip assembly, comprising:
a capillary tube having an open distal end;
an optical waveguide positioned within the capillary tube, wherein the optical waveguide causes guided light entering a proximal end of the optical waveguide to spread and to have a larger angular divergence at a distal end compared to the angular divergence of the entering guided light at the proximal end;
the optical waveguide being recessed from the open distal end of the capillary tube; and
a gap formed between the optical waveguide and the capillary tube, wherein the gap is configured to draw a liquid into the capillary tube toward the optical waveguide via a capillary effect.

2. The laser tip assembly of claim 1, wherein the capillary tube is constructed of a metal.

3. The laser tip assembly of claim 1, wherein the optical waveguide is a single crystal optical fiber.

4. The laser tip assembly of claim 1, wherein the optical waveguide is made of a material selected from sapphire or silica-based materials.

5. The laser tip assembly of claim 3, wherein the single crystal optical fiber has a hexagonal cross-section.

6. The laser tip assembly of claim 3, wherein the single crystal optical fiber has an oblong cross-section.

7. The laser tip assembly of claim 1, wherein the optical waveguide is tapered.

8. The laser tip assembly of claim 1, wherein the optical waveguide has a rounded distal end.

9. The laser tip assembly of claim 1, wherein the capillary tube is closed at a proximal end to limit the capillary effect.

10. The laser tip assembly of claim 9, further comprising an adhesive closing the proximal end of the capillary tube.

11. The laser tip assembly of claim 1, wherein the capillary tube comprises a metal that is thin enough to bend with the optical waveguide.

12. The laser tip assembly of claim 5, wherein an inner surface of the capillary tube contacts one or more vertices of the single crystal optical fiber.

13. The laser tip assembly of claim 1, wherein the gap extends from the open distal end of the capillary tube to a proximal end of the capillary tube, or the capillary tube includes an aperture to expose the gap to ambient pressure.

14. The laser tip assembly of claim 1, wherein the larger angular divergence of the guided light as it exits the distal end of the optical waveguide provides a safety mechanism such that if no liquid is present in the capillary tube, the spreading light materially contacts an inner surface of the capillary tube rather than surrounding tissue.

15. A method of providing laser energy via a laser tip assembly, comprising:
providing a capillary tube having an open distal end;
positioning an optical waveguide within the capillary tube, wherein the optical waveguide causes guided light to increase its angular divergence by the time it exits the distal end of the optical waveguide;
recessing the optical waveguide from the open distal end of the capillary tube; and
providing a gap formed between the optical waveguide and the capillary tube, wherein the gap is configured to draw a liquid into the capillary tube toward the optical waveguide via a capillary effect.

16. The method of claim 15, further comprising positioning the laser tip assembly adjacent a treatment area including a liquid.

17. The method of claim 16, wherein positioning the laser tip assembly comprises bending the capillary tube with the optical waveguide.

18. The method of claim 15, further comprising drawing the liquid into the capillary tube toward the optical waveguide.

19. The method of claim 15, further comprising delivering a laser energy through the optical waveguide into the liquid within the capillary tube.

20. The method of claim 19, further comprising creating a cavitation bubble in the liquid with the laser energy to at least one of (i) break up a soft tissue or hard tissue, or (ii) provide disinfection.
